(19) United States
(12) Patent Application Publication
(10) Pub. No.: US 2010/0069837 A1
(43) Pub. Date: Mar. 18, 2010

(54) Balloon Assembly and Method for Therapeutic Agent Delivery

(75) Inventors: Jay Rassat, Buffalo, MN (US); Derek Sutermeister, Eden Prairie, MN (US); James Anderson, Fridley, MN (US); Benjamin Arcand, Minneapolis, MN (US)

Correspondence Address: VIDAS, ARRETT & STEINKRAUS, P.A., SUITE 400, 6640 SHADY OAK ROAD, EDEN PRAIRIE, MN, 55344, US

(73) Assignee: Boston Scientific Scimed, Inc., Maple Grove, MN (US)

(21) Appl. No.: 12/211,528

(22) Filed: Sep. 16, 2008

(51) Int. Cl. A61M 25/10

(52) U.S. Cl. 604/99.04; 604/103.01

(57) ABSTRACT

A balloon assembly includes an elongated balloon, one or more cutting elements engaged to the balloon, and one or more therapeutic agents disposed within an opening defined by the cutting blades. The balloon assembly converts energy into heat to enhance elution of the therapeutic agent at the treatment site

### CROSS-REFERENCE TO RELATED APPLICATIONS

Not Applicable

### STATEMENT REGARDING FEDERALLY SPONSORED RESEARCH

Not Applicable

### BACKGROUND OF THE INVENTION

1. Field of the Invention

Embodiments of the present invention pertain generally to medical catheters and balloons More particularly, some embodiments of the present invention pertain to balloon assemblies for treating lesions in the human vasculature.

2. Description of the Related Art

Balloon assemblies having surface features suitable for treating lesions are viewed by many as the next generation treatment option for the revascularization of both coronary and peripheral vessels, can be used as a replacement for conventional percutaneous transluminal coronary angioplasty (PTCA) procedures. Such balloon assemblies are described in commonly assigned U.S. Pat Nos. 7,070,576 and 7,153,315, as well as in commonly assigned and co-pending U.S. Patent Application Nos. 2005/0119678 and 2006/0116700, the entire contents of each being expressly incorporated herein by reference

The art referred to and/or described above is not intended to constitute an admission that any patent, publication or other information referred to herein is “prior art” with respect to this invention. In addition, this section should not be construed to mean that a search has been made or that no other pertinent information as defined in 37 C F R §1.56(a) exists.

All U S patents and applications and all other published documents mentioned anywhere in this application are incorporated herein by reference in their entirety.

Without limiting the scope of the invention, a brief summary of some of the claimed embodiments of the invention is set forth below. Additional details of the summarized embodiments of the invention and/or additional embodiments of the invention may be found in the Detailed Description of the Invention below.

A brief abstract of the technical disclosure in the specification is provided for the purposes of complying with 37 C.F.R §172.

### BRIEF SUMMARY OF THE INVENTION

In at least one embodiment, the invention is directed to a balloon assembly for use on a catheter to incise tissue at a treatment site in a body vessel The balloon assembly includes an elongated balloon, one or more cutting elements, and one or more therapeutic agents. The balloon is disposed about a longitudinal axis and has an unexpanded state and an expanded state The one or more cutting elements are engaged to the balloon. Each cutting element defines an opening which is at least partially loaded with one or more therapeutic agents. The balloon assembly converts energy into heat to enhance elution of the therapeutic agent at the treatment site.

Cutting elements, as the phrase is used herein, includes any structure that incises, penetrates, scores, cuts, etc. tissue including, but not limited to, blades, wires, protrusions, barbs, and/or similar structures

In some embodiments of the present invention, one or more of the cutting elements comprise two dissimilar metals joined together at at least one region.

In at least one embodiment, the balloon assembly further comprises a piezoelectric material. In some embodiments, the piezoelectric material is engaged to the cutting element, and the cutting element is comprised of a conductive material In at least one embodiment, the piezoelectric material is selected from the group consisting of polyvinylidene difluoride and lead-zirconia-titania

In some embodiments, the balloon assembly further comprises electroactive metals

In some embodiments, the invention is directed to a balloon assembly for use on a catheter to treat tissue at a treatment site in a body vessel. The balloon assembly comprises an elongated balloon, one or more pores, one or more therapeutic agents, and one or more thermodynamic valves The balloon is disposed about a longitudinal axis and has an unexpanded state and an expanded state. The therapeutic agent(s) are disposed within the pores. The valve(s) are constructed and arranged to open thereby releasing the therapeutic agent(s).

In at least one embodiment, the thermodynamic valve comprises a bimetallic actuator.

In some embodiments, the thermodynamic valve(s) comprise one or more cutting blades

These and other embodiments which characterize the invention are pointed out with particularity in the claims annexed hereto and forming a part hereof However, for further understanding of the invention, its advantages and objectives obtained by its use, reference should be made to the drawings which form a further part hereof and the accompanying descriptive matter, in which there is illustrated and described embodiments of the invention.

### BRIEF DESCRIPTION OF THE SEVERAL VIEWS OF THE DRAWING(S)

A detailed description of the invention is hereafter described with specific reference being made to the drawings.

FIG. 1 is a simplified, perspective view of a catheter having a balloon assembly operationally positioned in the body of a patient.

FIG. 2 is an enlarged, perspective view of a balloon assembly, in accordance with at least one embodiment of the present invention

FIG. 3 is a cross-sectional view of the balloon assembly shown in FIG. 2, as seen along line 3-3 in FIG. 2.

FIG. 4 is an enlarged, perspective view of an incising element of the balloon assembly shown in FIG. 2, in accordance with at least one embodiment of the present invention.

FIG. 5 is an enlarged, cross-sectional view of a balloon assembly, in accordance with at least one embodiment of the present invention.

FIG. 6 is an enlarged, perspective view of a balloon assembly, in accordance with at least one embodiment of the present invention.

FIG. 7 is an enlarged, side view of an incising element of the balloon assembly shown in FIG. 6, in accordance with at least one embodiment of the present invention.

FIG. 8 is an enlarged, cross-sectional view of an incising element with dissimilar metals, in accordance with at least one embodiment of the present invention.

FIG. 9 is an enlarged, cross-sectional view of an incising element combined with a piezoelectric material, in accordance with at least one embodiment of the present invention

FIG. 10 is an enlarged, cross-sectional view of the incising element of FIG. 9, shown with the piezoelectric material compressed, in accordance with at least one embodiment of the present invention.

FIG. 11 is an enlarged, cross-sectional view of an incising element combined with a radiant energy source, in accordance with at least one embodiment of the present invention.

FIG. 12 is an enlarged, side view of a balloon assembly with pores, in accordance with at least one embodiment of the present invention

FIG. 13 is an enlarged, cross-sectional view of a balloon assembly with a pore, with a closed thermodynamic valve, in accordance with at least one embodiment of the present invention.

FIG. 14 is an enlarged, cross-sectional view of a balloon assembly with a pore, with an open thermodynamic valve, in accordance with at least one embodiment of the present invention.

FIG. 15 is an enlarged, cross-sectional view of a balloon assembly with a pore, with an open thermodynamic valve with incising elements, in accordance with at least one embodiment of the resent invention.

FIG. 16 is an enlarged, cross-sectional view of a balloon assembly, in accordance with at least one embodiment of the present invention.

FIG. 17 is an enlarged, side view of a balloon assembly, in accordance with at least one embodiment of the present invention.

FIG. 18 is an enlarged, cross-sectional view of a balloon assembly, in accordance with at least one embodiment of the present invention.

### DETAILED DESCRIPTION OF THE INVENTION

While this invention may be embodied in many different forms, there are described in detail herein specific preferred embodiments of the invention. This description is an exemplification of the principles of the invention and is not intended to limit the invention to the particular embodiments illustrated.

For the purposes of this disclosure, like reference numerals in the figures shall refer to like features unless otherwise indicated

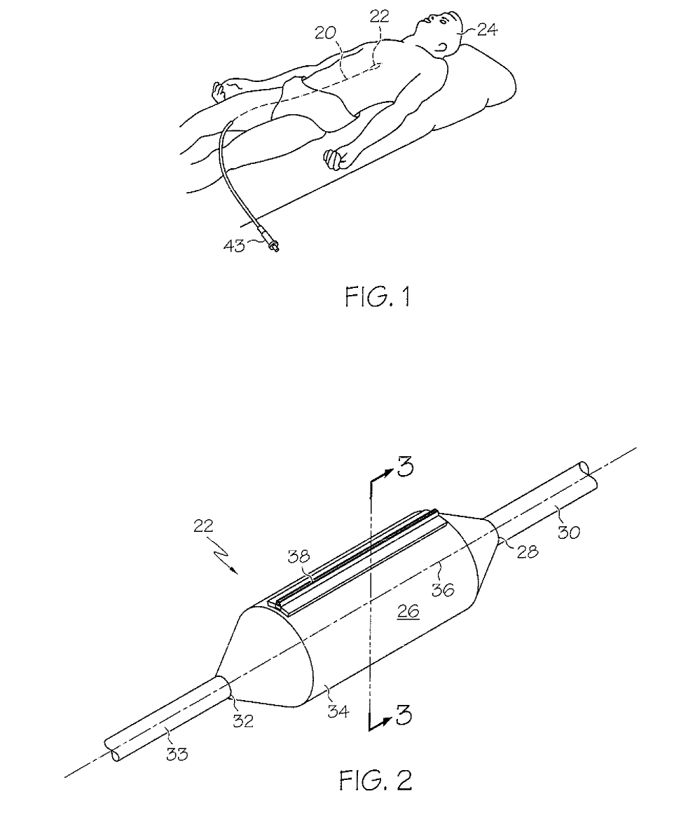

Referring initially to FIG. 1, a catheter 20 having a balloon assembly 22 is shown for performing a medical procedure at an internal treatment site of a patient 24 More specifically, the catheter 20 is shown positioned to treat a lesion in a body artery. Although the catheter 20 is capable of performing a medical procedure in a body artery such as a coronary artery, those skilled in the pertinent art will quickly recognize that the use of the catheter 20 as herein described is not limited to use in a specific artery, but, instead can be used in vascular conduits and other ductal systems throughout the human body.

Turning now to FIG. 2, the distal portion of the catheter 20 of FIG. 1 is shown to include a balloon assembly 22 having an inflatable balloon 26. Inflatable balloon 26 has a distal end 28 attached to a distal tube 30 and a proximal end 32 attached to a proximal tube 33. FIG. 2 further shows that the inflatable balloon 26 typically includes a cylindrically shaped working section 34 disposed about a balloon axis 36. Typically, the inflatable balloon 26 is made of a polymeric material such as polyethylene terephthalate (PET) or nylon. Examples of other materials suitable for use as the inflatable balloon can be found in U.S. Pat. No. 7,070,576, the entire content of which is expressly incorporate herein by reference

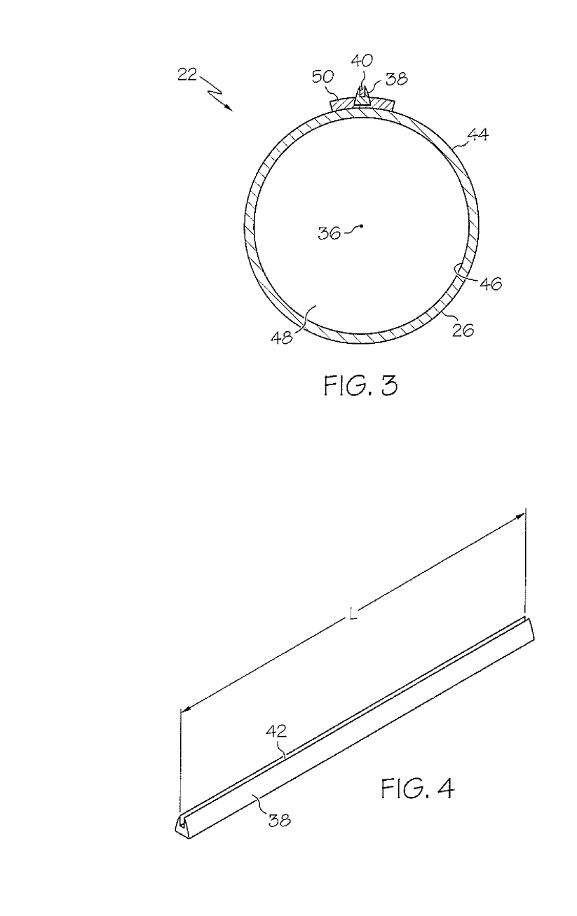

Referring again to FIG. 2, the balloon assembly 22 further includes one or more cutting elements 38, such as blades, wires, or members. In some embodiments the wire may be a helical wire. The thickness of the wire can be varied in order to control the depth of penetration into the tissue, as desired. In the embodiment depicted in FIG. 2, the cutting element 38 is an elongated blade that extends radially outward (relative to the balloon axis 36). As best seen in FIG. 3, the blade 38 is designed to store a therapeutic agent 40 Specifically, the elongated blade is designed with a slot 42 extending along at least a portion of the length L of the blade, as shown in FIG. 4 While the embodiment shown in FIG. 4 depicts a slot extending along the entire length of the blade, the slot can extend only partially along the length of the blade. The blade can be divided into three regions: two end regions and an intermediate region between the two end regions In at least one embodiment, there is a slot only in the intermediate region. In some embodiments, there is a slot in only one end region. In at least one embodiment, there is a slot in both end regions.

Referring again to FIG. 3, the inflatable balloon 26 can be characterized as having an outer surface 44 and an opposed inner surface 46 that surrounds an inflation lumen 48 that can be infused with a medical grade fluid (not shown) to expand the inflatable balloon 26 More specifically, as shown in FIG. 1, an inflation device, which for the embodiment shown is a syringe 43, can be activated to pump a medical grade fluid through the inflation tube to expand the inflatable balloon 26. FIG. 3 depicts the blade 38 embedded in a blade pad 50 which is used to facilitate attachment of the blade to the outer surface 44 of the inflatable balloon 26.

As mentioned early, the slot acts as a reservoir for one or mole therapeutic agents that are loaded into the blade prior to delivery. As the balloon is inflated, the blade is driven into the tissue to be treated, thereby also delivering the therapeutic agent directly to the treatment site.

The agent can be in the form of a coating or other layer (or layers) of material, a powder, or a crystal, each adapted to be released at the site of the balloon's implantation or areas adjacent thereto A therapeutic agent can be a drug or other pharmaceutical product such as non-genetic agents, genetic agents, cellular material, etc. Some examples of suitable non-genetic therapeutic agents include but are not limited to: anti-thrombogenic agents such heparin, heparin derivatives, prostaglandin (including micellar prostaglandin El), urokinase, and PPack (dextrophenylalanine proline arginine chloromethylketone); anti-proliferative agents such as enoxaparin, angiopeptin, sirolimus (rapamycin), tacrolimus, everolimus, zotarolimus, monoclonal antibodies capable of blocking smooth muscle cell proliferation, hirudin, and acetylsalicylic acid; anti-inflammatory agents such as dexamethasone, rosiglitazone, prednisolone, corticosterone, budesonide, estrogen, estrodiol, sulfasalazine, acetylsalicylic acid, mycophenolic acid, and mesalamine; anti-neoplastic/anti-proliferative/anti-mitotic agents such as paclitaxel, epothilone, cladribine, 5-fluorouracil, methotrexate, doxorubicin, daunorubicin, cyclosporine, cisplatin, vinblastine, vincristine, epothilones, endostatin, trapidil, halofuginone, and angiostatin; anti-cancer agents such as antisense inhibitors of c-myc oncogene; anti-microbial agents such as triclosan, cephalosporins, aminoglycosides, nitrofurantoin, silver ions, compounds, or salts; biofilm synthesis inhibitors such as non-steroidal anti-inflammatory agents and chelating agents such as ethylenediaminetetraacetic acid, O,O′-bis (2-aminoethyl) ethyleneglycol-N,N,N′,N′-tetraacetic acid and mixtures thereof, antibiotics such as gentamycin, rifampin, minocyclin, and ciprofloxacin; antibodies including chimeric antibodies and antibody fragments; anesthetic agents such as lidocaine, bupivacaine, and ropivacaine; nitric oxide; nitric oxide (NO) donors such as linsidomine, molsidomine, L-arginine, NO-carbohydrate adducts, polymeric or oligomeric NO adducts; anti-coagulants such as D-Phe-Pio-Arg chloromethyl ketone, an RGD peptide-containing compound, heparin, antithrombin compounds, platelet receptor antagonists, anti-thrombin antibodies, anti-platelet receptor antibodies, enoxaparin, hirudin, warfarin sodium, Dicumarol, aspirin, prostaglandin inhibitors, platelet aggregation inhibitors such as cilostazol and tick antiplatelet factors; vascular cell growth promotors such as growth factors, transcriptional activators, and translational promotors; vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents; vasodilating agents; agents which interfere with endogenous vascoactive mechanisms; inhibitors of heat shock proteins such as geldanamycin; angiotensin converting enzyme (ACE) inhibitors; beta-blockers; βAR kinase (βARK) inhibitors; phospholamban inhibitors; protein-bound particle drugs such as ABRAXANE®; and any combinations and prodrugs of the above.

Exemplary biomolecules include peptides, polypeptides and proteins; oligonucleotides; nucleic acids such as double or single stranded DNA (including naked and cDNA), RNA, antisense nucleic acids such as antisense DNA and RNA, small interfering RNA (siRNA), and ribozymes; genes; carbohydrates; angiogenic factors including growth factors; cell cycle inhibitors; and anti-restenosis agents. Nucleic acids may be incorporated into delivery systems such as, for example, vectors (including viral vectors), plasmids or liposomes.

Non-limiting examples of proteins include serca-2 protein, monocyte chemoattractant proteins (MCP-1) and bone morphogenic proteins (“BMPs”), such as, for example, BMP-2, BMP-3, BMP-4, BMP-5, BMP-6 (VGR-1), BMP-7 (OP-1), BMP-8, BMP-9, BMP-10, BMP-11, BMP-12, BMP-13, BMP-14, BMP-15. Preferred BMPs are any of BMP-2, BMP-3, BMP-4, BMP-5, BMP-6, and BMP-7. These BMPs can be provided as homodimers, heterodimers, or combinations thereof, alone or together with other molecules. Alternatively, or in addition, molecules capable of inducing an upstream or downstream effect of a BMP can be provided. Such molecules include any of the “hedghog” proteins, or the DNA's encoding them. Non-limiting examples of genes include survival genes that protect against cell death, such as anti-apoptotic Bcl-2 family factors and Akt kinase; serca 2 gene; and combinations thereof. Non-limiting examples of angiogenic factors include acidic and basic fibroblast growth factors, vascular endothelial growth factor, epidermal growth factor, transforming growth factors α and β, platelet-derived endothelial growth factor, platelet-derived growth factor, tumor necrosis factor α, hepatocyte growth factor, and insulin-like growth factor. A non-limiting example of a cell cycle inhibitor is a cathespin D (CD) inhibitor. Non-limiting examples of anti-restenosis agents include p15, p16, p18, p19, p21, p27, p53, p57, Rb, nFkB and E2F decoys, thymidine kinase and combinations thereof and other agents useful for interfering with cell proliferation.

Exemplary small molecules include hormones, nucleotides, amino acids, sugars, and lipids and compounds have a molecular weight of less than 100 kD.

Exemplary cells include stem cells, progenitor cells, endothelial cells, adult cardiomyocytes, and smooth muscle cells. Cells can be of human origin (autologous or allogenic) or from an animal source (xenogenic), or genetically engineered. Non-limiting examples of cells include side population (SP) cells, lineage negative (Lin−) cells including Lin⁻CD34⁻, Lin⁻CD34⁺, Lin⁻cKit⁺, mesenchymal stem cells including mesenchymal stem cells with 5-aza, cold blood cells, cardiac or other tissue derived stem cells, whole bone marrow, bone marrow mononuclear cells, endothelial progenitor cells, skeletal myoblasts or satellite cells, muscle derived cells, go cells, endothelial cells, adult cardiomyocytes, fibroblasts, smooth muscle cells adult cardiac fibroclasts+5-aza genetically modified cells, tissue engineered grafts, MyoD scar fibroblasts, pacing cells, embryonic stem cell clones, embryonic stem cells, fetal or neonatal cells, immunologically masked cells, and teratoma derived cells.

Any of the therapeutic agents may be combined to the extent such combination is biologically compatible. Further, each of the plurality of vesicles on the medical devices of the present invention can contain a single therapeutic agent or multiple therapeutic agents Further, the plurality of vesicles can collectively contain the same therapeutic agents or at least some different therapeutic agents.

In embodiments of a medical device having a coating, such a coating can be biodegradable or non-biodegradable. Non-limiting examples of suitable non-biodegradable polymers include metals or metallic oxides; polystrene; polyisobutylene copolymers, styrene-isobutylene block copolymers such as styrene-isobutylene-styrene tri-block copolymers (SIRS) and other block copolymers such as styrene-ethylene/butylene-styrene (SEBS); polyvinylpyrrolidone including cross-linked polyvinylpyrrolidone; polyvinyl alcohols, copolymers of vinyl monomers such as EVA; polyvinyl ethers; polyvinyl aromatics; polyethylene oxides; polyesters including polyethylene terephthalate; polyamides; polyacrylamides; polyethers including polyether sulfone; polyalkylenes including polypropylene, polyethylene and high molecular weight polyethylene; polyumethanes; polycarbonates, silicones; siloxane polymers; cellulosic polymers such as cellulose acetate; polymer dispersions such as polyurethane dispersions (BAYHDROL®); squalene emulsions; and mixtures and copolymers of any of the foregoing.

Non-limiting examples of suitable biodegradable polymers include polycarboxylic acid, polyanhydrides including maleic anhydride polymers; polyorthoesters; poly-amino acids; polyethylene oxide; polyphosphazenes; polylactic acid, polyglycolic acid and copolymers and mixtures thereof such as poly(L-lactic acid) (PLLA), poly(D,L,-lactide), poly(lactic acid-co-glycolic acid), 50/50 (DL-lactide-co-glycolide); polydioxanone; polypropylene fumarate; polydepsipeptides; polycaprolactone and co-polymers and mixtures thereof such as poly(D,L-lactide-co-caprolactone) and polycaprolactone co-butylacrylate; polyhydroxybutyrate valerate and blends; polycarbonates such as tyrosine-derived polycarbonates and arylates, polyiminocarbonates, and polydimethyltrimethylcarbonates; cyanoacrylate; calcium phosphates; polyglycosaminoglycans; macromolecules such as polysaccharides (including hyaluronic acid; cellulose, and hydroxypropylmethyl cellulose; gelatin; starches; dextrans; alginates and derivatives thereof), proteins and polypeptides; and mixtures and copolymers of any of the foregoing. The biodegradable polymer may also be a surface erodable polymer such as polyhydroxybutyrate and its copolymers, polycaprolactone, polyanhyduides (both crystalline and amorphous), maleic anhydride copolymers, and zinc-calcium phosphate.

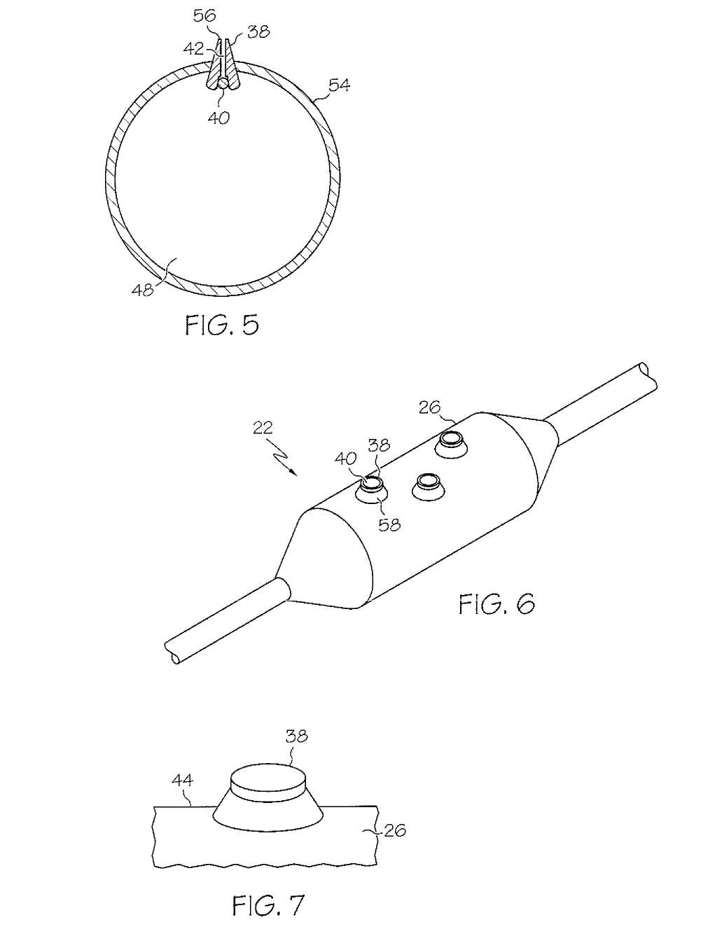

While the design of FIG. 3 shows the therapeutic agent stored in the blade and exterior to the balloon, another embodiment of the invention is directed towards storing one or more therapeutic agents within a cutting element reservoir located within the interior of the balloon, as best seen in FIG. 5. In FIG. 5, the base 52 of the cutting element 38, or blade as it is depicted in FIG. 5, extends through the balloon material 54 and into the inflation lumen 48. The blade is designed with a cavity 42 that extends from the base 52 of the blade through the tip 56 of the blade. The opening of the cavity at the base of the blade is located within the inflation lumen 48. As before, the cavity is loaded with one or more therapeutic agents 40. In such an embodiment, as the injection or inflation fluid is forced into the balloon, the therapeutic agent mixes with fluid and is forced out through the tip of the blade to the site of treatment

The cutting elements can also be one or more elliptical, or in the embodiment shown in FIG. 6, circular blades 38 In such an embodiment, rather than creating a slot within the blade to load with the therapeutic agent(s), at least some of the region defined by and within the perimeter 58 of the blade is instead loaded with the therapeutic agent(s) 40. FIG. 7 depicts a close up of the circular blade 38

Referring now to FIG. 8, the cutting element 38 can be comprised of two or more dissimilar metals joined together. Dissimilar metals joined together generate a voltage and do not provide actuation. One metal comprises the cathode and the other metal comprises the anode. The electrolytic solution contained between the two dissimilar metals permits the transport of energy due to the differences in electric potential (voltage) existing between the two metals FIG. 8 shows the cutting element 38 made of a first metal M1 and a second metal M2 When placed in contact with an electrolyte (such as in blood), the metals form a galvanic couple that creates an electric potential, and resultingly, a current flow between the metals as they begin to corrode The cutting element 38, as described above, is loaded with one or more therapeutic agents 40 The current created by the joining of the dissimilar metals heats the cutting element and serves to activate the therapeutic agent(s) for improved drug delivery. The further apart the metals are on the standard electrode potential table, the more current will flow. A non-limiting list of metals that can be used include iron, magnesium, calcium, and zinc

As stated earlier, some embodiments of the system may utilize electroactive metals (EAMs) such as nitinol (nickel-titanium alloy) instead of or in addition to the use of dissimilar metals. EAMS ate materials which actuate due to current, voltage, or heat. That is, the EAMS move or change shape, typically in response to current or other electrical interaction, for example heat. This can be used to manipulate the balloon assembly blades In at least one embodiment, the cutting features are not exposed during delivery, but actively exposed at a treatment site. These cutting features (blades, wires, protrusions, etc.) can be folded down on their side or embedded into the balloon. Alternate embodiments may include maintaining low delivery profiles with proper wrapping of blades and balloon, or blade movement to improve cutting action

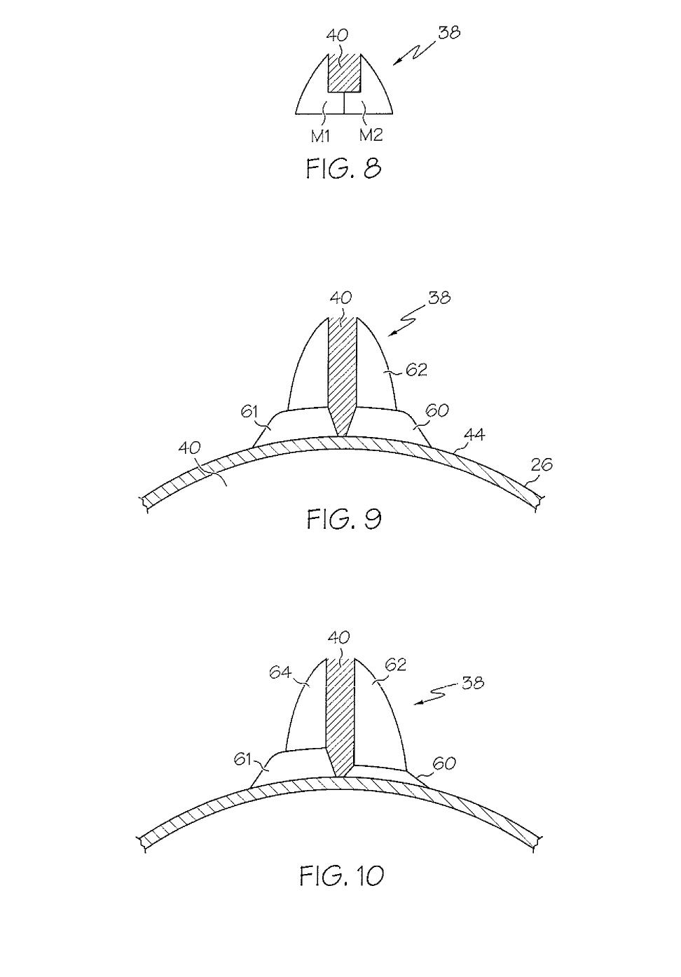

Rather than relying on the use dissimilar metals to create a current flow as in FIG. 8, the embodiment in FIG. 9 relies on piezoelectricity. Piezoelectricity is the ability of certain materials to generate an electric potential upon application of a mechanical force. FIG. 9 depicts a layer of piezoelectric material 60 between a first portion 62 of the cutting element 38 and the balloon 26 As the balloon 26 is inflated, the exterior 44 of the balloon compresses the layer of piezoelectric material 60 against the first portion 62 of the incising element 38. A voltage V is generated by the piezoelectric material, as shown in FIG. 10, and exists at first portion 62. The second portion 64 of the cutting element remains at a different potential than first portion 62 because material 61 does not exhibit piezoelectric behavior. The two portions of the cutting element 38 ate separated by the therapeutic agent(s) 40. In some embodiments, the therapeutic agent forms a conductive path between the first portion and the second portion, thereby allowing a small current to flow and activate the therapeutic agent(s) for improved drug delivery. Due to shape or pressure changes in its molecular structure, the piezoelectic will generate a current. Any suitable piezoelectric materials can be used including, but not limited to lead zirconate titanate (or “PZT”) and polyvinylidene difluoride (or “PVDF”).

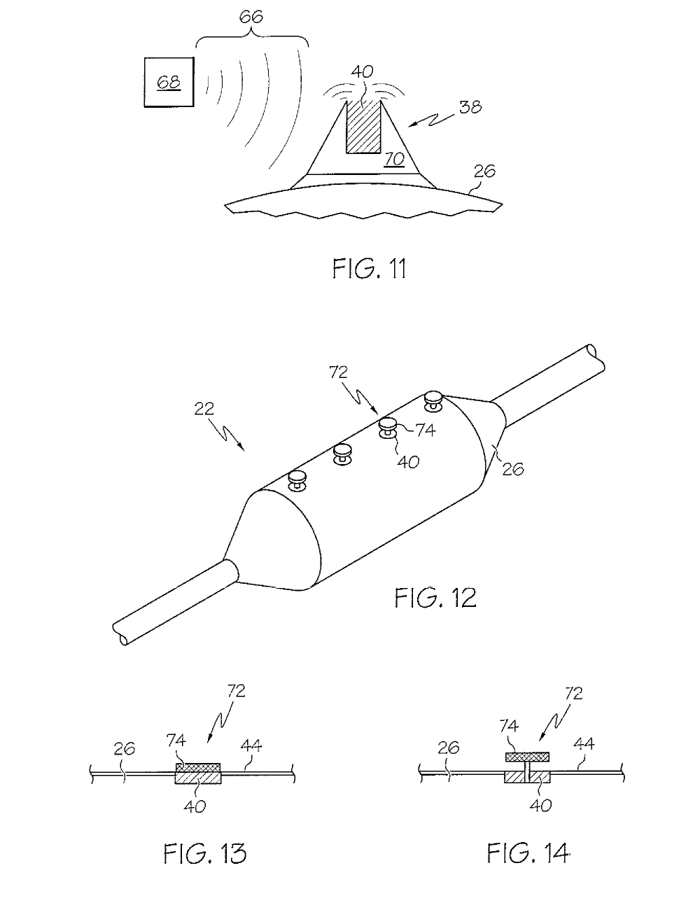

Referring now to FIG. 11, a cutting element 38 of a balloon is shown that vibrates in response to the application of radiant energy 66 from radiant energy source 68. The radiant energy 66 can be in the form of radiofrequency (RF) waves. More specifically, the radiant energy can be in the ultrasonic frequency range or microwave frequency range. As the RF waves strike the conductive material 70 of the cutting elements, the waves induce an electric current in the metal. The metal has some resistance, therefore the induced current will heat the incising elements. As before, the cutting elements are loaded with therapeutic agent(s) 40 and the heat serves to activate the therapeutic agent(s) for improved drug delivery

Other methods of employing radiant energy, piezoelectric material, electric potential, or electrolytes can be found in U.S. Pat. No. 6,656,162, the entire contents of which is incorporated herein by reference

Another embodiment of the present invention is depicted in FIG. 12. Although FIG. 12 depicts pores 72 embedded within the wall of a balloon, the pores can also be embedding within the struts of a stent. FIG. 13 is a close-up of the pore in FIG. 12. In FIG. 13, the pore is loaded with a therapeutic agent(s) 40 A bimetallic valve 74 covers the pore and substantially seals in the therapeutic agent 40 prior to delivery, thereby preventing the release of the therapeutic agent until the balloon has been delivered to the treatment site. Once delivered to the treatment site, the bimetallic valve 74 opens as a result of the different expansion characteristics of the two metals, as seen in FIG. 14. The open valve allows the release of the therapeutic agent(s) at the treatment site.

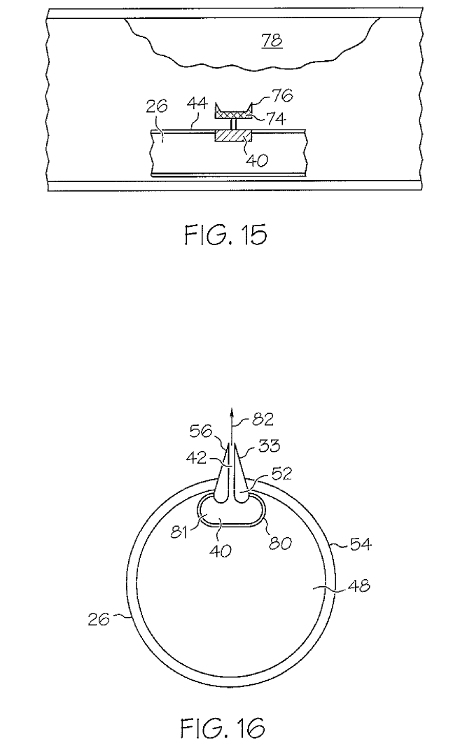

FIG. 15 depicts the embodiment of FIG. 14 with the addition of incising elements 76 on the bimetallic valve 74. In such an embodiment, the valve opens at the treatment site, as described above. Once open, the incising elements 76 cut into the stenosis 78 and the therapeutic agent(s) 40 are delivered directly to the treatment site. Direct delivery to the treatment site improves the efficacy of the drug treatment because greater quantities of the drugs reach the treatment site.

In some embodiments of the invention, a pathway from a valve-style manifold is used to inject the liquid or gel through the catheter and into the body through the incising elements. In such an embodiment, a tube 80 with lumen 81 is disposed within the inflation lumen 48 of the inflatable balloon 26, as seen in FIG. 16. The base 52 of the cutting element 38, or blade as it is depicted in FIG. 16, extends through the balloon material 54 and into the inflation lumen 48. The blade is designed with a cavity 42 that extends from the base 52 of the blade through the tip 56 of the blade. The base 52 of the cutting element is engaged to the tube 80 Each cavity 42 is in communication with the lumen 81 of the tube 80. Tube 80 is injected with a therapeutic agent 40 in a liquid or gel form which proceeds through the tube and out the cavity 42, as indicated by arrow 82, to the treatment site. It should be noted that the pathway is distinct from the inflation lumen that inflates the balloon.

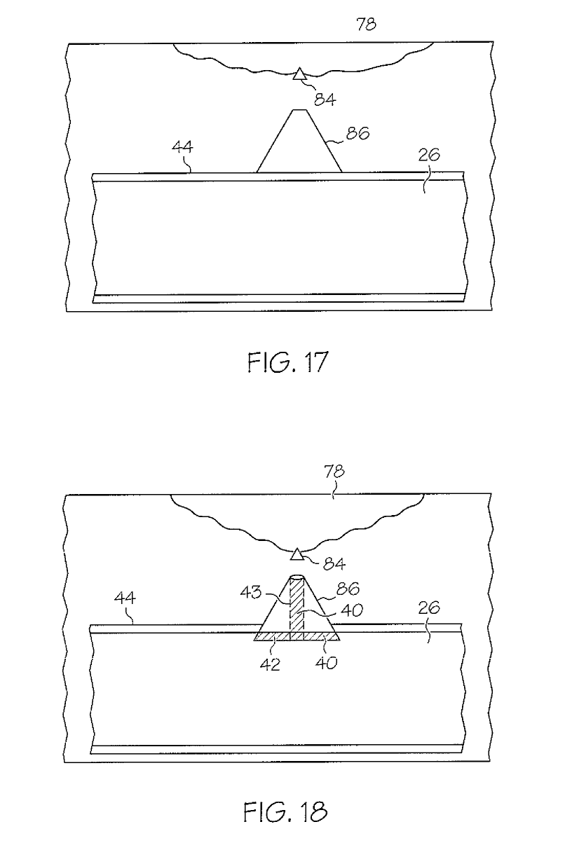

In at least one embodiment, the surface of the balloon, or the struts of a stent, can include barbs, needles, micro-tubules, or other objects (hereafter collectively referred to as “barbs”) for depositing or etching therapeutic agents into a stenosis. In an unexpanded condition, the balloon assembly with the barbs passes easily through a body lumen without depositing or etching therapeutic agent. However, as shown in the embodiment in FIG. 17, when the balloon 26 is delivered to the treatment site and expanded, the tip 84 of a barb 86 becomes embedded within the stenosis 78 and breaks off. Upon breaking off, the tip 84 releases a therapeutic agent(s) directly into the stenosis. The barb itself can be made of the therapeutic agent, such as in crystallized form, as in FIG. 17.

Or, in other embodiments, the tip 84 of the barb 86 can act as a cap on a cavity or reservoir 42 containing a therapeutic agent(s) 40 within the remaining barb, as seen in FIG. 18. When the tip is broken off, a pathway 43 is created from the reservoir 42 through the remaining barb to the stenosis, thereby allowing the therapeutic agent 40 contained within the reservoir to be delivered directly to the stenosis 78. In some embodiments, the barb is left embedded within the stenosis. In at least one embodiment, the barbs are removed from the stenosis after a certain period of time.

FIGS. 17 and 18 are also directed toward an embodiment employing thermostat drug release This is a pore or drug releasing member that activates when the body undergoes a change in temperature. For example, an inflammatory response can increase the body temperature above a threshold of the heat-activated pore. When activated, the pore delivers therapeutic agents to the site to aid/speed the recovery of the affected area.

In some embodiments, the balloon can also include a tube adjacent to the balloon for fluid delivery of a therapeutic agent(s). The tube can be preloaded with a drug, or the tube can be used as a pathway to deliver a drug to the treatment site. The tube delivers the drug to the treatment as a result of the deployment of the balloon.

In some embodiments the cutting element, the balloon, the delivery system or other portion of the assembly can include one or more areas, bands, coatings, members, etc. that is (are) detectable by imaging modalities such as X-Ray, MRI, ultrasound, etc. In some embodiments at least a portion of the stent and/or adjacent assembly is at least partially radiopaque.

The above disclosure is intended to be illustrative and not exhaustive. This description will suggest many variations and alternatives to one of ordinary skill in this art The various elements shown in the individual figures and described above may be combined or modified for combination as desired. All these alternatives and variations are intended to be included within the scope of the claims where the term “comprising” means “including, but not limited to”.

Further, the particular features presented in the dependent claims can be combined with each other in other manners within the scope of the invention such that the invention should be recognized as also specifically directed to other embodiments having any other possible combination of the features of the dependent claims For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

This completes the description of the preferred and alternate embodiments of the invention. Those skilled in the art may recognize other equivalents to the specific embodiment described herein which equivalents are intended to be encompassed by the claims attached hereto.

What is claimed is:

1. A balloon assembly for use on a catheter, the balloon assembly comprising:
an elongated balloon, the balloon disposed about a longitudinal axis, the balloon having an unexpanded state and an expanded state;
at least one cutting element, the at least one cutting element engaged to the balloon, the at least one cutting element defining an opening; and
at least one therapeutic agent, the therapeutic agent disposed within the opening defined by the at least one cutting blade,
wherein the balloon assembly converts energy into heat to enhance elution of the therapeutic agent at the treatment site.

2. The balloon assembly of claim 1, wherein the at least one cutting element comprises two dissimilar metals joined together at at least one region

3. The balloon assembly of claim 2, further comprising an electrolytic solution

4. The balloon assembly of claim 1, further comprising a piezoelectric material

5. The balloon of claim 4, wherein the piezoelectric material is engaged to the cutting element, the cutting element comprised of a conductive material.

6. The balloon assembly of claim 4, wherein the piezoelectric material is selected from the group consisting of polyvinylidene difluoride and lead-zirconia-titania.

7. The balloon assembly of claim 1, further comprising at least one electroactive metal

8. The balloon assembly of claim 7, wherein the at least one electroactive metal is nitinol

9. A balloon assembly for use on a catheter, the balloon assembly comprising:
an elongated balloon, the balloon disposed about a longitudinal axis, the balloon having an unexpanded state and an expanded state;
at least one pore;
at least one therapeutic agent, the therapeutic agent disposed within the at least one pole;
at least one thermodynamic valve, wherein the at least one valve has a closed state and an open state, and wherein the at least one valve releases the at least one therapeutic agent when in the open state.

10. The balloon assembly of claim 9, wherein the thermodynamic valve comprises a bimetallic actuator.

11. The balloon assembly of claim 9, wherein the at least one thermodynamic valve comprises at least one cutting blade.
